(19) United States
(12) Patent Application Publication
(10) Pub. No.: US 2024/0299698 A1
(43) Pub. Date: Sep. 12, 2024

(54) PREVENTION AND TREATMENT OF POST-OPERATIVE COGNITIVE DYSFUNCTION (POCD)

(71) Applicant: Noetix Pharma LLC, Bethesda, MD (US)

(72) Inventor: Arnold L. Newman, Bethesda, MD (US)

(21) Appl. No.: 18/133,664

(22) Filed: Apr. 12, 2023

Related U.S. Application Data
(60) Provisional application No. 63/336,455, filed on Apr. 29, 2022.

(51) Int. Cl. A61M 21/00; A61K 31/4418; A61P 25/28

(52) U.S. Cl. CPC A61M 21/00; A61K 31/4418; A61P 25/28; A61M 2021/0077

(57) ABSTRACT

A method for the prevention and/or treatment of post-operative cognitive dysfunction, which entails administering to a human or other mammal an effective amount of one or more compounds selected from the group consisting of N-substituted 2(1H) pyridones, N-substituted 3(1H) pyridones and pharmaceutically-acceptable salts of any one or more of the above pyridones, which are optionally further substituted at various available ring positions.

### BACKGROUND OF THE INVENTION

The term perioperative neurocognitive disorder describes behavior, affect, and cognition disorders that can occur after anesthesia and surgery. It can include preexisting cognitive impairment, postoperative delirium, and neurocognitive impairment (e.g., delayed neurocognitive recovery or neurocognitive disorder-postoperative).

Post-Operative Cognitive Dysfunction (POCD) is a neurocognitive impairment brought on by surgery performed under general anesthesia and diagnosed by the comparison of pre-operative and post-operative neurocognitive domain testing. It is characterized by a decline in a patient's cognition or activities of daily living. A delay in recovery can last up to thirty days. A neurocognitive disorder is one that lasts longer than thirty days, up to a year or even longer. POCD has been associated with the worsening or onset of dementia and this can be observed within five years following surgery. (Mahanna-Gabrielli, E. et al.) (All documents cited herein are incorporated fully in their entirety by reference. Use of these references does not imply that they are common knowledge or an indication of the state of the art.)

An international study of POCD in the elderly (60 years or older), published in 1998 (Moller et al.), found that POCD was present in 25.8% of patients one week after surgery. Three months following surgery, POCD had persisted in 9.9% of patients. Furthermore, it was found that increasing age, anesthesia duration, low education level, a second operation, post-operative infections and respiratory problems put people at risk for early POCD. Only age was found to be a factor for late POCD. (Moller J T, et al.)

According to Kitsis et al., there is no specific treatment for POCD. Current treatment involves minimizing the risk to the patient by careful selection of anesthesia, addressing modifiable risk factors, and using interventions such as particular pain management strategies, vital sign monitoring, adequate fluid and oxygen administration and daily cognitive stimulation sessions for 6 days, etc. (Kitsis P, et al.)

Since the Moller et al. study, various investigators have confirmed the significance of POCD with various incidences reported depending on the type of surgery and neurocognitive tests performed. Unfortunately, there is, to date, still an unmet need for a pharmaceutical approach to the prevention of POCD.

### SUMMARY OF THE INVENTION

Accordingly, it is an object of the present invention to provide a method preventing a neurocognitive disorder brought on by surgery.

Furthermore, it is an object of the present invention to provide a method of preventing post-operative cognitive dysfunction in a patient following an operation on said patient.

It is also an object of the present invention to ameliorate the cognitive dysfunction resulting from surgery performed under anesthesia.

It is further an object of the present invention to provide a method wherein a POCD preventing pharmaceutical agent is administered to a patient in an amount sufficient to prevent said cognitive dysfunction in said patient.

Another object of the present invention is to provide a method wherein a POCD ameliorating agent is administered to a patient pre-operatively to prevent post-operative cognitive impairment.

A further object of the invention is to provide a method wherein a pharmaceutical agent that works against post-operative cognitive dysfunction is administered to a patient both pre-operatively and post-operatively.

It is, additionally, an object of the invention to administer pirfenidone or a pirfenidone analog or derivative to a patient in advance of an operation with a dose that prevents the onset of post-operative cognitive dysfunction.

It is further an object of the invention to administer pirfenidone or a pirfenidone analog or derivative to a patient before surgery with a dose that ameliorates post-operative cognitive dysfunction.

It is, moreover, an object of the invention to administer pirfenidone or a pirfenidone analog or derivative to a patient before and after an operation with doses that prevent the onset of post-operative cognitive dysfunction.

It is further an object of the invention to administer pirfenidone or a pirfenidone analog or derivative to a patient before and after surgery with doses that ameliorate post-operative cognitive dysfunction.

It is yet another object of the invention to provide for an agent for preventing or reducing cognitive decline in a patient wherein said agent is pirfenidone, a pirfenidone derivative, a pirfenidone analog, or a combination of two or more of pirfenidone, a pirfenidone derivative or pirfenidone analog.

The above objects and others discussed below are provided by a method of preventing and ameliorating post-operative cognitive dysfunction, which entails administering an amount of pirfenidone, or pirfenidone analog, or pirfenidone derivative one or more times before an operation, during an operation, after an operation, or any combination thereof to the human patient. The analogs and derivatives of pirfenidone are described herein.

### BRIEF DESCRIPTION OF THE DRAWINGS

FIG. 1 is a bar graph showing fear conditioning context testing results in terms of time spent freezing on post-surgical days 1 and 3.

FIG. 2 is a bar graph that illustrates the overall percent time spent freezing of mice during fear conditioning cued testing.

FIG. 3 is a bar graph of the time spent freezing during fear conditioning cued testing for pre-tone, tone, and post-tone time points.

FIG. 4 is a bar graph of novel object recognition exploration times during training and at 1 hour and 24 hours following training for each experimental group

FIG. 5 is a bar graph comparing novel object recognition preferences at 1 hour and 24 hours post-training for each experimental group.

FIG. 6 is a bar graph that depicts the mean time to platform comparing block 1 and block 2 of each experimental group during Morris water maze testing.

FIG. 7 is a bar graph that shows the mean time to platform of the Morris water maze testing wherein the experimental groups are shown within a block.

FIG. 8 is a bar graph that depicts Morris water maze mean time in quadrant probe trial data.

FIG. 9 is a non-parametric evaluation that shows the effect on the morphological characteristics of hippocampal microglial cells of treated vs. control animals.

### DETAILED DESCRIPTION OF THE INVENTION AND EMBODIMENTS

### Term Definitions

Cognitive dysfunction—refers to deficits in attention, verbal and non-verbal learning, short term and working memory, visual and auditory processing, problem solving and processing speed.

Cytokine inflammatory cascade—proinflammatory cytokines play a central role in inflammatory disease of infectious or noninfectious origin; examples of which are IL-1, IL-6, IL-8, IL-12, IL-18 and TNF.

Gliosis—Is the excessive development of glia especially interstitially. It is a nonspecific reactive change of glial cells in response to damage to the central nervous system. Most often, gliosis involves the proliferation or hypertrophy of several different types of glial cells, including astrocytes, microglia, and oligodendrocytes. (Wikipedia) It also typically involves changes in cell phenotype evidenced by alterations in cellular morphology.

Neuroinflammation—inflammation of a nerve or of parts of the nervous system that resulting from the activation of the excessive secretion and/or presence of pro-inflammatory cytokines.

Operation—an act of surgery performed on a patient. It can include but is not limited to surgery performed relating to the central nervous system, peripheral nervous system, endocrine organs and ectopic tumors, eye, ears, respiratory system, cardiovascular system, lymphatic system, gastrointestinal system and mouth, urinary tract, male and female reproductive systems, bone, cartilage and joints, muscle and other soft tissue, the breast, skin and other miscellaneous anatomical areas.

Systemic inflammation—Systemic inflammation is defined as “typical, multi-syndrome, phase-specific pathological process, developing from systemic damage. For the purposes of the present invention, systemic inflammation is a general response olf the body to the trauma of surgery.

TNF-α, IL-1b, and IL-6—are cytokines that play important roles in inflammatory processes.

The trauma of an operation can have a profound effect upon the body and especially the brain. One common but significant complication resulting from surgical trauma is manifested in a decline of cognition following an operation. Although it can happen at any age, the incidence and severity of cognitive decline following surgery increases with age.

The complex processes that cause POCD are not fully understood. However, some studies have presented the hypothesis that neuroinflammation is a factor that relates to cognitive decline after surgery. (Safavynia S A, et al., Berger M, et al.) There is also a hypothesis that neuroinflammation may be a factor associated with gliosis (e.g., microgliosis, astrogliosis), which, in combination with neuroinflammation, may have a role in POCD etiology, but this association (e.g., whether correlative or causative) and the putative mechanisms involved are uncertain. These and other factors may possibly be components of the pathological processes leading to cognitive decline following surgery, which is also known as post-operative cognitive dysfunction (POCD). (Wan Y, et al., Terrando N, et al.)

I hypothesized that pirfenidone (5-methyl-1-phenyl-2-(1H)-pyridone) might be useful for preventing the onset of POCD or reducing its effects. Pirfenidone is an inhibitor, in various tissues, of inflammatory cytokines, particularly TNF-α, which may be involved in the initiation of POCD. (Cain W C, et al., Oku H, et al.) It is a non-steroidal organic molecule (i.e., a non-biologic), a modified pyridone with a molecular weight of 185.22 and, thus, a relatively small molecule compared to most biologics. Biologic inhibitors of cytokines such as antibodies or modified receptor fragments are able to inhibit systemic inflammation but they are too large to cross the blood brain barrier. Thus, any effect they might have on neuroinflammation would appear to be indirect. In contrast, pirfenidone can cross the blood brain barrier and thus might be able to suppress inflammation both systemically and in the brain. (Macias-Barragán J, et al.) To test this hypothesis, a series of experiments, discussed below, were devised and undertaken to investigate the use of pirfenidone against POCD in a mouse model.

The project included experiments performed to investigate the therapeutic effects of administration of pirfenidone in a mouse model of (POCD). The model was developed during pilot work completed earlier in the study and was induced by performing a laparotomy with a 5-minute manipulation of abdominal viscera and musculature. Weights and physical condition of the animals were closely monitored throughout the study, with presurgical and daily post-surgical gavage of pirfenidone or saline administered. Behavioral testing was performed to evaluate alterations in learning and memory processes using fear conditioning, novel object recognition, and water maze paradigms. Following euthanasia on the eighth day post-surgery, hippocampal tissue was preserved and later evaluated for morphological characteristics related to microglial cell activation.

The study protocol was reviewed and approved by the animal ethics committee of the Atlantic Veterinary College. All animal husbandry and experimental procedures were in conformance with the guidelines established by the Canadian Council for Animal Care (http://www.ccac.ca/en_/standards/guidelines)

Upon arrival, mice were group housed (4 per cage) in an SPF facility and allowed a minimum of 7 days for acclimation to the facility and investigators prior to initiation of test article administration. During this acclimation period, mice were handled to minimize stress during study activities. All mice were maintained on a standard 12-hour day/night lighting schedule (06:00 h-18:00 h), with temperature and relative humidity maintained at 22-23° C. and 40-60% respectively. Standard rodent chow (Lab Diet 5001) and reverse osmosis water was provided ad libitum.

Mice were weighed 24 hours after arrival, weekly prior to the initiation of pirfenidone administration, and then daily throughout the remainder of the study. Post-induction, animals were assessed daily to monitor body condition, pain response, and general recovery parameters.
Test System: Male C57BI/6 mice
Number of Animals/Group: 12 animals per group (total of 48 mice)
Age: Cohort 1: 12 weeks at arrival
Cohort 2: 10 weeks at arrival
Model Induction: Performed at 14 weeks of age
Weight: 31.6±0.42 SEM g at time of surgery
Allocation: Animals were pseudo-randomly assigned to treatment groups balanced for weight
Health inspection: A health inspection was performed by a clinical veterinarian prior to the commencement of the study.

### Test Articles

Test articles consisted of pirfenidone that was orally administered (i.e., gavage) to adult mice 10 minutes prior to model induction at a dose level of 400 mg/kg and then one per day throughout the remainder of the study at 200 mg/kg (multi), or at a single time point (ten minutes) prior to model induction at 400 mg/kg (acute). To balance the treatment groups and eliminate potential confounds, mice in the acute group received saline only at all remaining time points equivalent to those used for the multi-dose group.

### Treatment Groups:

A. Laparotomy+Pirfenidone (Chronic; 400 mg/kg initial; 200 mg/kg p.o. 1×/day throughout remainder of study)
B. Laparotomy+Pirfenidone (Acute; 400 mg/kg; single presurgical dose. Saline p.o. 1×/day throughout remainder of study)
C. No surgery+saline (Normal Control; Saline p.o. 1×/day for study duration)
D. Laparotomy+saline (Disease Control; Saline p.o. 1×/day for study duration)

### Controls

Both normal (Group C) and disease (Group D) controls were used in this study.

Normal control mice received no model induction but were administered saline in an identical manner and volume to that used for the test article.

Negative control (i.e., disease control) mice underwent model induction surgery and received an equivalent volume of saline delivered in an identical manner to that used for the test article.

TABLE 1
Summary of Treatment Groups

| | | | Group |
|---|---|---|---|
| Group | Treatment | Purpose | Number |
| A | Induction + Pirfenidone (400 | Test article - multi | 12 |
| | mg/kg initial; 200 mg/kg QD | | |
| | throughout remainder of study) | | |
| B | Induction + Pirfenidone (400 | Test article - acute | 12 |
| | mg/kg) | | |
| C | No induction + saline | Normal Control | 12 |
| D | Induction + saline | Negative Control | 12 |

Pirfenidone doses and administered saline were prepared as follows:

### High Dose Information

Identity: Pirfenidone
Source: TCI America
Appearance: White powder
Storage conditions: Stored protected from light at room temperature (20-25°)

### High Dose Preparation

Vehicle: 0.9% saline
Working concentration: 66.67 mg/ml
Preparation: Add 1000 mg of pirfenidone to 15 mL of 0.9% saline
Stability in vehicle: Stable for a minimum of 8 days

### High Dose Administration

Administration method: Oral gavage
Dose: 6 mL/kg per mouse; equivalent to 400 mg/kg.
Dose Frequency: Acute; once prior to surgery for treatment groups A and B.
Dose Duration: Beginning 10 minutes prior to model induction and continuing until euthanasia

### Low Dose Information

Identity: Pirfenidone
Source: TCI America
Appearance: White powder
Storage conditions: Stored protected from light at room temperature (20-25°)

### Low Dose Preparation

Vehicle: 0.9% saline
Working concentration: 33.33 mg/mL
Preparation: Add 12 mL of high dose pirfenidone to 12 mL of 0.9% saline
Stability in vehicle: Stable for a minimum of 8 days

### Low Dose Administration

Administration method: Oral gavage
Dose: 6 mL/kg per mouse; equivalent to 200 mg/kg.
Dose Frequency: Daily; Treatment group B only.
Dose Duration: 8 Days

### Model Preparation

### Laparotomy

To induce POCD, mice were anesthetized with isoflurane and maintained at a surgical plane of anesthesia (2-2.5%). The abdomen was shaved and cleaned/disinfected with both betadine and ethanol (70%). A 1.5 cm vertical incision was made along the midline of the abdomen, perforating the skin and the peritoneal cavity. A sterile blunt probe was inserted into the cavity, and the abdominal viscera and musculature were manipulated for five minutes. Abdominal muscles and skin were sutured, polysporin and topical lidocaine were applied to the incision site, and buprenorphine (0.1 mg/kg s.c.) was administered. Animals were placed into a clean, pre-warmed home cage for recovery and housed singly thereafter.

### Control Animals

Disease control (DC) mice underwent laparotomy surgery and received an equivalent volume of saline delivered in an identical manner to that used for test article groups. Normal control (NC) mice did not undergo any surgical procedures but were single housed in an equivalent manner to surgery groups for the remainder of the study to avoid confounds from housing and received an equivalent volume of saline delivered in an identical manner to that used for test article groups.

### Physical Assessments

### Weight

Mice were weighed 24 hours after arrival, weekly prior to the initiation of Test Article administration, and then daily throughout the remainder of the study.

No weight difference between groups was present at surgery (Normal Control: 31.7±0.62 g; Disease Control: 31.5±79 g; Pirfenidone Acute dose 31.6±1.1 g; Pirfenidone Chronic dose 31.8±0.92 g). While all mice lost some weight at 24 hours post-surgery, this loss did not differ between groups and all mice continued to gain and/or maintain weight through the following week.

### Pain and General Health

Pain and general health were assessed daily in all animals. Incision sites were checked for signs of infection or disruption of stitches. Animals that experienced weight loss ≥1 g, or otherwise seemed impaired, were provided with a wet food slurry to encourage weight gain and reduce dehydration until there was a return to normal physiology.

### Behavioral Testing

Behavioral testing was performed at various time points throughout the study as outlined in each separate section below and summarized in Table 2. All tests were recorded and analyzed by experimenters blinded to treatment. Data were obtained through a combination of experimenter scoring and use of AnyMaze™ Video Tracking System evaluation.

TABLE 2
Behavioral test battery for assessment of general
health status and cognitive function.

| | Time of | |
|---|---|---|
| Test | Testing | Measure |
| Fear Conditioning (FC) | 24 hrs prior to | Trace conditioning |
| Training | surgery | |
| Context Testing (FC) | PSD1, 3 | Associative memory - |
| | | context |
| Cued Testing (FC) | PSD 3 | Associative memory - |
| | | cued |
| Morris Water Maze | PSD7; probe on | Spatial learning & memory; |
| | PSD8 | reference memory |
| Novel Object | PSD5-6 | Recognition memory |
| Recognition | | |

PSD = post-surgical day.

### Fear Conditioning Testing

Fear conditioning is a commonly used test of associative learning where a neural stimulus is paired with an aversive unconditioned stimulus, resulting in a freezing response that can be subsequently quantified. Training occurred on the day prior to surgery, context testing on PSD1 (i.e., 2 days after training) and PSD3 (i.e., 4 days after training), and cued testing on PSD3 at a time point three hours after the final context test. For training and context testing, mice were placed in an operant chamber (Med Associates). For cued testing, mice were placed into a novel arena with novel external environment and scent.

### Fear Conditioning: Training

Fear conditioning training was performed to generate an association between a neutral “conditional” stimulus (80 dB tone) with an aversive “unconditional” stimulus (0.4 mA shock). Before training, the system was tested to confirm proper operation of the shock and tone. During training, the testing environment was kept quiet and well controlled. Temperature and humidity levels were kept constant and the experimenter was not visible from the operant chamber, which was cleaned between animals. The training protocol was as follows: 1. Each mouse was habituated to the testing room for 20 minutes. 2. Video recording was initiated and the animal's ID card was recorded. 3. Each mouse was placed into the operant chamber and the training protocol was implemented as follows: a. 120 seconds; no stimuli (habituation to the chamber); b. 20 seconds, tone presentation (80 dB); c. 20 seconds, no stimuli (trace delay period); d. 2 seconds, foot shock (0.4 mA); e. 90 seconds, no stimuli (intertrial interval); f. repeat steps a-d followed by a final 30 second no stimuli period. 4. The mouse was returned to its home cage.

### Fear Conditioning: Context Testing

The purpose of context testing is to evaluate context memory for an aversive environment. Materials for this process included the operant testing chamber, a video recorder, a stopwatch, and data recording sheets. During the testing process the testing environment was kept quiet and well controlled with constant temperature and humidity levels. It was ensured that the experimenter was not visible from the operant chamber. The chamber was cleaned between animals.

Context testing was performed as follows: 1. The mouse was habituated to the testing room for 15 minutes. 2. Video recording was initiated and the animal's ID card recorded. 3. The mouse was placed in the operation chamber and allow ed 270 seconds exploration time. 4. The mouse was returned to its home cage.

### Fear Conditioning: Cued Testing

The purpose of cued testing is to evaluate cued memory for an aversive stimulus. Materials for this process included the novel chamber with a speaker, dedicated computer and software, external cues and scented (vanilla) cotton ball, a video recorder and data recording sheets.

Prior to testing, the test system (e.g., computer, tone, etc.) was checked for proper performance. External cue visibility was checked and the freshly scented cotton ball was placed inside the arena. The testing environment was kept quiet and well controlled and constant temperature and humidity levels were maintained. The operator was not visible from the operant chamber, which was cleaned between animals. This test did not require that the animals be habituated to the room.

Cued testing was performed as follows: 1. The animal was brought into the testing room in a transfer cage. 2. Video recording was initiated and the animal's ID card was recorded. 3. The mouse was placed into the novel chamber and the cued testing protocol on the computer was started and implemented as follows: a. 135 seconds; no stimuli (pre-tone phase); b. 135 seconds; the tone was presented (80 dB); c. 30 seconds; no stimuli (post-tone phase). 4. The mouse was returned to its home cage.

### Fear Conditioning Results

Fear conditioning context testing was performed on PSD1 and PSD3, with exposure to the contextual environment presented at the equivalent time of day to the training period in the same arena as was used for training. On PSD3, following the final contextual testing, the cued testing phase was performed. This test was performed in a distinct chamber to that used for training and contextual testing, with differences in chamber walls, floor, and scent, but with the same tone presentation. The primary dependent measure for these test sessions was time spent freezing.

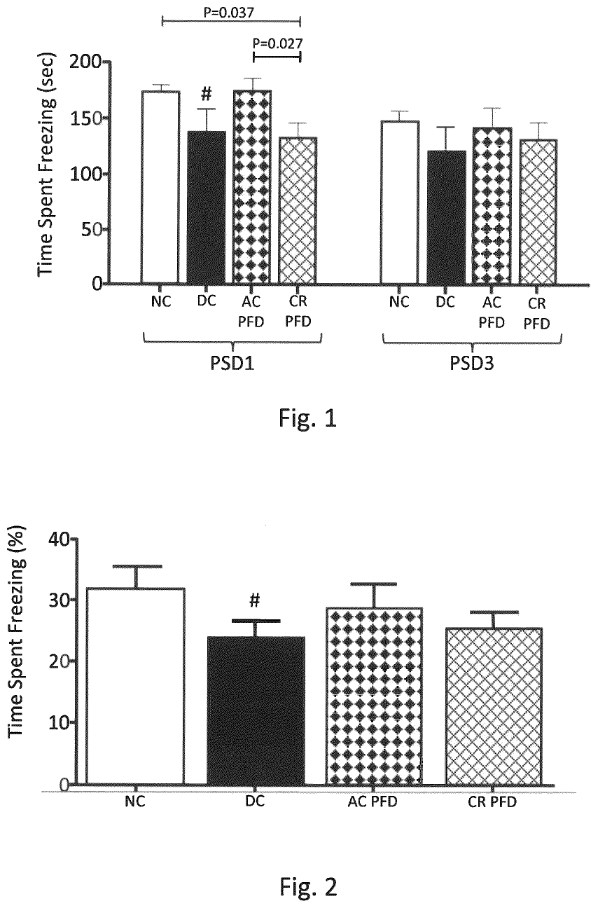

Contextual fear conditioning results are shown graphically in FIG. 1, which shows the time spent freezing, in seconds, for each test group. The test groups are denoted as follows: Normal Controls: NC; Disease Controls: DC; Acute pirfenidone administration: AC PFD; Chronic pirfenidone administration: CR PFD. Error bars denote SEM. Normal Control (NC) n=11, Disease Control (DC) n=11, Acute pirfenidone (AC PFD) n=12, Chronic pirfenidone (CR PFD) n=12.

In the contextual test on PSD1 (i.e., two days after training), a significant, main effect was noted between groups (p=0.042, One-way ANOVA with Welch correction) during the 270 second test period. An evaluation with LSD post-hoc was performed and revealed a trend toward a difference between Normal and Disease Controls (see #sign; p=0.078; Disease Controls spent less time freezing), whereas both Normal Control and Acute pirfenidone groups exhibited increased freezing (indicative of better contextual memory for an aversive environment) compared to Chronic pirfenidone animals (p=0.037 and p=0.027 respectively).

On PSD3, no statistical difference between groups was evident (p=0.671, One-way ANOVA). Interestingly, although within-group evaluation revealed that both Normal Control and Acute pirfenidone treated mice spent less time freezing in the context chamber during this second test presentation compared to freezing time on PSD1 (paired t-tests; p=0.020 and p=0.009 respectively with a trend toward less freezing noted in Disease Controls, p=0.063), Chronic pirfenidone mice exhibited no difference between test presentation days (p=0.942; paired t-test). It is important to note that less freezing time on the second presentation day is typically expected, as repeated exposure to the same context in the absence of an aversive stimulus often results in some extinction of the fear response. In this case, it is unclear why no extinction effect was present in the Chronic pirfenidone treated animals. However, it should be noted that Acute pirfenidone treated mice once again performed equivalently to Normal Controls on PSD3.

To summarize the results of this experimental model, in which mice were trained 24 hours before surgery and tested on PSD1 and PSD3, the most significant results were evident on PSD1, but those of PSD3 still showed a noticeable trend.

FIG. 2 depicts the results of fear conditioning cued testing (overall measures), which was performed on PSD3 (i.e., four days after training). Fear conditioning cued testing was evaluated at 3 discrete time points: prior to the tone, during the tone, and following the tone. While no main effect was present at any timepoint (p>0.05, one-way ANOVA), mean freezing times trended toward a decrease in Disease Controls (see #sign) compared to Normal Controls overall (p=0.075, t-test), and Chronic pirfenidone administered mice performed more similarly to Disease Control. Acute pirfenidone administered mice exhibited increased mean performance (i.e., longer freeze times) compared to both the Chronic pirfenidone treated mice and the Disease controls. In FIG. 2, error bars denote SEM. Normal Control (NC) n=11, Disease Control (DC) n=11, Acute pirfenidone (AC PFD) n=12, Chronic pirfenidone (CR PFD) n=12.

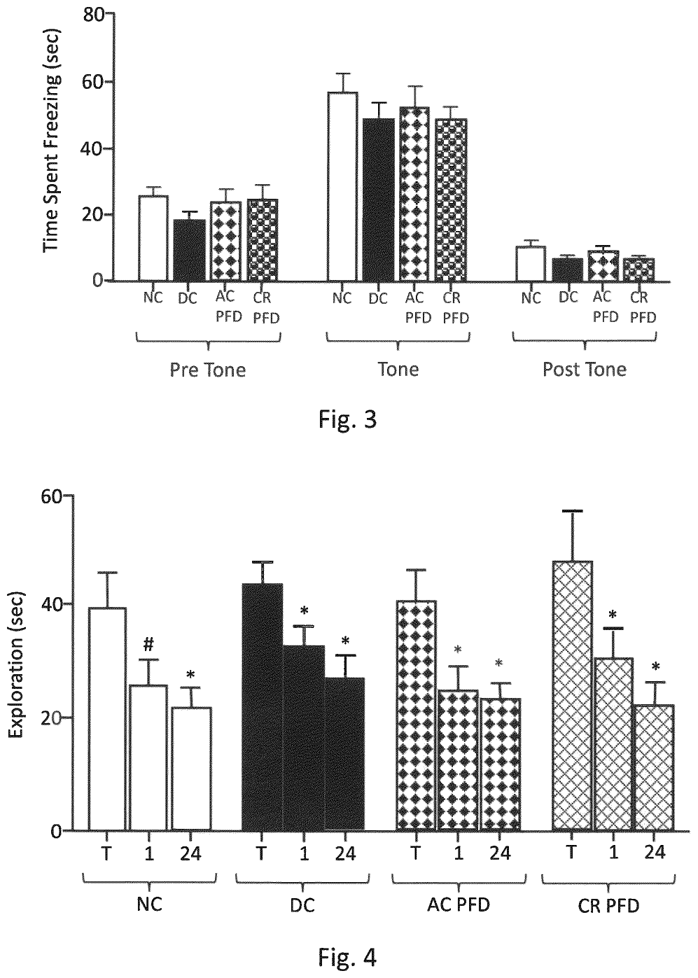

FIG. 3 is a graph that shows the time spent freezing for the pre-tone, tone, and post-tone times. Within-group evaluation comparing freezing response in the pre-tone timepoint vs. freezing during tone presentation and freezing during tone presentation compared to freezing post-tone was statistically significant for all groups (p<0.01, paired t-test), indicating that all mice still had a strong aversive association for the tone.

The post-tone period is the one that corresponds closely with trace conditioning, as mice learn during training that the shock occurs following the tone, and thus this is the period during which it would be most anticipated. Here, a trend toward decreased freezing times (and thus decreased memory) was noted for both Disease Controls and Chronic pirfenidone treated mice compared to Normal Control animals (see #signs—Disease vs Normal, p=0.098; Chronic pirfenidone vs. Normal Controls, p=0.088). Acute pirfenidone mice exhibited increased mean performance (i.e., longer freeze times) compared to Disease Control mice. In FIG. 3, error bars denote SEM. Normal Control (NC) n=11. Disease Control (DC) n=11, Acute pirfenidone (AC PFD) n=12, Chronic pirfenidone (CR PFD) n=12.

### Novel Object Recognition Testing

Novel object recognition (NOR) is a tool used to assess the ability of mice to differentiate between familiar and novel objects (i.e., recognition memory). The paradigm relies on the tendency of rodents to interact more with a novel object than a familiar one. In this task, acclimation to the test arena was provided on the day prior to training (PSD4) and the initial test trial (short-term recall), presented one hour following training on PSD5. The second test trial (long-term recall) was performed 24 hours later (PSD6). Object recognition may be determined through evaluation of the percent time spent exploring a novel object relative to the total time spent exploring both novel and familiar objects; normal animals will spend more time exploring a novel object. If the exploration of both objects is the same, this behavior can be a sign of a recognition memory deficit.

### Novel Object Recognition: Acclimation

The purpose of acclimation was to allow mice to explore and become familiar with the testing area. Materials used included: 1. Arena (50 cm×50 cm). 2. Webcam/computer (AnyMaze™ software). 3. Stopwatch. 4. Data recording sheets.

The following were general considerations: 1. A quiet, consistently lit testing room was ensured. 2. The arena was cleaned prior to the acclimation of each animal. 3. No objects were put into the arena for this phase.

The acclimation process was as follows: 1. The animal was brought into the testing room in a transfer cage. 2. Video recording was begun and the animal's ID card was recorded. 3. The mouse was placed in the center of the arena and allowed to explore freely for 5 minutes. 4. The mouse was returned to its home cage.

### Novel Object Recognition: Training

The purpose of NOR training was to familiarize mice with two identical objects. Materials used included: 1. Arena (50 cm×50 cm). 2. Webcam/computer (AnyMaze™ software). 3. Two objects (rectangular blue blocks). 4. Stopwatch. 5. Data recording sheets.

The following were general considerations: 1. A quiet, consistently lit testing room was ensured. 2. The arena was cleaned prior to the acclimation of each animal. 3. Each object was attached at the designated position in the arena.

The paired object training process was as follows: 1. The animal was brought into the testing room in a transfer cage. 2. Video recording was begun and the animal's ID card was recorded. 3. The mouse was placed in the center of the arena and allowed to explore freely for 10 minutes. 4. The mouse was returned to its home cage.

### Novel Object Recognition: Test 1

The purpose of this test was to evaluate short-term (1 hour) recognition memory. Materials used included: 1. Arena (50 cm×50 cm) 2. Webcam/computer (AnyMaze™ software). 3. Familiar objects (rectangular blue block). 4. Novel objects (pink ball). 4. Stopwatch. 5. Data recording sheets.

General considerations of this protocol were as follows: 1. A quiet, consistently lit testing room was ensured. 2. The arena was cleaned prior to the acclimation of each animal. 3. Animals were tested one hour after training. 4. Object locations were rotated between trials to eliminated place preference effects.

The novel object (1 hour) test was performed as follows: 1. The arena was prepared with one original object (block) and one novel object (ball) present. 2. The animal was brought into the testing room in a transfer cage. 3. Video recording was begun and the animal's ID card was recorded. 4. The mouse was placed in the center of the arena and allowed to explore for 10 minutes. 5. The mouse was returned to its home cage.

### Novel Object Recognition: Test 2

The purpose of this test was to evaluate long-term (24 hour) recognition memory. Materials used included: 1. Arena (50 cm×50 cm). 2. Webcam/computer (AnyMaze™ software). 3. Familiar objects (rectangular blue block). 4. Novel objects (metal dome). 4. Stopwatch. 5. Data recording sheets.

General considerations of this test were as follows: 1. A quiet, consistently lit testing room was ensured. 2. The arena and objects were cleaned between each animal. 3. Each individual animal was tested at the same time each day. 4. Object locations were rotated between trials to eliminated place preference effects.

The novel object (24 hour) test was performed as follows: 1. The arena was prepared with one original object (block) and one novel object (dome) present. 2. The animal was brought into the testing room in a transfer cage. 3. Video recording was begun and the animal's ID card was recorded. 4. The mouse was placed in the center of the arena and allowed to explore for 10 minutes. 5. The mouse was returned to its home cage.

### Novel Object Recognition Results

In the novel object recognition test, all mice regardless of treatment exhibited similar overall exploration patterns (training, p=0.823; 1-hr test, p=0.603; 24-hr test, p=0.767; one-way ANOVA), spending the most total time interacting with the identical objects during the training session. During novel object testing 1 hour after training, all treatment groups showed a decrease in total time spent interacting with objects overall compared with training (Normal Control, p=0.06; Disease Control, p=0.03; Acute pirfenidone, p=0.01; Chronic pirfenidone, p=0.04; within group paired t-test). While exploration times decreased again 24 hours later (training vs 24-hour exploration times, p<0.05; within group paired t-test), the difference between 1-hour and 24-hour exploration was not statistically significant within any treatment group. Exploration time data are shown in FIG. 4, which, for each experimental group, are provided for testing time points at training (T), one hour (1) and twenty-four hours (24).

In FIG. 4, a trend toward a difference compared to training in the Normal Control group is indicated by the #sign, while a statistically significant difference for test times compared to the within group training time is denoted by an asterisk. Error bars denote SEM. Normal Control (NC) n=12, Disease Control (DC) n=11, Acute Pirtenidone (AC PFD) n=12, Chronic pirfenidone (CR PFD) n=12.

For evaluation of object preference, mice that did not meet the minimum criteria for exploration time were excluded from analysis, as it could not be confirmed that they spent enough time exploring to learn or discriminate.

While no main effect for treatment was present at the 1-hour (short term) testing time point (p=0.376, one-way ANOVA), a treatment effect was noted for 24-hour (long term) testing (p=0.054). Post-hoc (LSD) testing showed a trend toward increased preference (i.e., increased memory) for both Acute and Chronic pirfenidone administered mice compared to Disease Controls (Acute vs Disease, p=0.08; Chronic vs Disease, p=0.09).

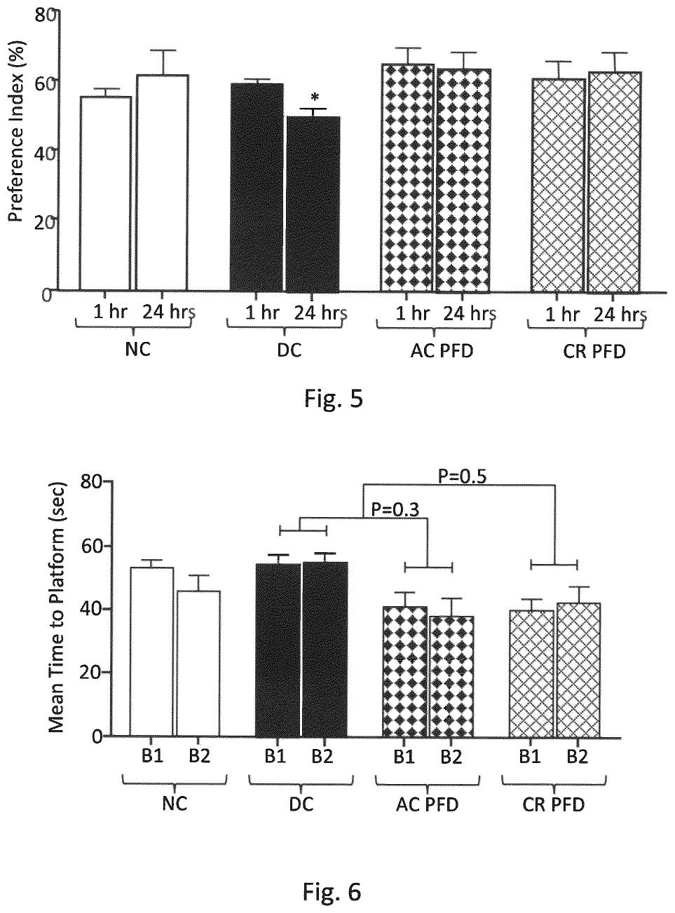

To further evaluate these trends, an assessment of within-group memory performance was run. These analyses revealed that all groups except Disease Control mice (p=0.017, paired t-test) retained memory for the familiar object as indicated by a statistically equivalent preference index between testing time points (Normal Control, p=0.477; Acute pirfenidone, p=0.762; Chronic pirfenidone, p=0.828; paired t-test). A graphical depiction of the 1-hour and 24-hour data is found in FIG. 5, which confirms that Disease Control mice overall experienced statistically decreased recognition memory between the 1-hour and 24-hour test periods (indicated by an asterisk), a difference that was not seen in any other treatment group. Error bars denote SEM. Normal Control (NC) n=9, Disease control (DC) n=11, Acute Pirfenidone (AC PFD) n=10, Chronic Pirfenidone (CR PFD) n=12.

### Morris Water Maze

Morris water maze testing is a well-established method for evaluating spatial learning and memory. To navigate this maze, the mouse must use extra-maze cues to locate a hidden platform and escape from the water. Testing was performed on PSD7. Mice were given two blocks of four 60 sec trials to find the hidden platform, with latency to reach the platform compared between blocks as a measure of learning/memory processes. Swim speed was evaluated to assess locomotor capabilities. On PSD8 (i.e., 24 hours later), mice were reintroduced into the maze without the platform present for a one-minute probe trial to assess reference memory.

The following materials were used during Morris Water Maze testing: 1. Water maze arena (96 cm diameter, 43 cm deep). 2. White, non-toxic paint (to make water opaque). 3. Escape platform. 4. Webcam/computer (AnyMaze™ software). 5. Stopwatch. 6. Data recording sheets.

General considerations relating to the protocol were as follows: 1. Pool was filled and emptied daily. 2. Water was maintained at a temperature of 22±1° C. 3. Water was cleaned and stirred between animals to eliminate scent cues. 4. It was ensured that the platform was submerged 1 cm below water level and positioned in the designated location.

The testing protocol was as follows: 1. The animal was brought into the testing room in a transfer cage. 2. Video recording was begun and the animal's ID card recorded. 3. The mouse was gently placed (i.e., not dropped) into the water at the appropriate start location (N, S, E, or W) facing the pool wall. 4. For 60 seconds, the mouse was allowed to locate and climb onto the submerged platform. If the mouse was unable to locate the platform within the allotted time, it was gently guided there and assigned a score of 61 seconds. 5. The mouse was allowed 60 seconds to recover on the platform and observe the extra-maze cues. If the moused jumped into the water, it was immediately retrieved and placed back on the platform for the remaining duration of the inter-trial interval. 6. Steps 3-5 were repeated for a total of 4 trials (Block 1). 7. The mouse was returned to the transfer cage lined with a dry towel. It was allowed 5 minutes to rest. 8 Steps 3-5 were repeated for a total of 4 trials (Block 2). 9. The mouse was gently dried and returned to its home cage.

The Morris Water Maze probe trial used the following materials: 1. Water maze arena (96 cm diameter, 43 cm deep). 2. White, non-toxic paint (to make water opaque). 3. Webcam/computer (AnyMaze™ software). 4. Stopwatch. 5. Data recording sheets.

General considerations of the probe trial included the following: 1. Pool was filled and emptied daily. 2. Water was maintained at a temperature of 22±1° C. 3. Water was cleaned and stirred between animals to eliminate scent cues. 4. No platform was used for this test.

The probe session was conducted as follows: 1. The animal was brought into the testing room in a transfer cage. 2. Video recording was begun and the animal's ID card recorded. 3. The mouse was gently placed (i.e., not dropped) into the water at the appropriate start location (N, S, E, or W) facing the pool wall. 4. The mouse was allowed to swim freely for 60 seconds. 5. The mouse was gently dried and returned to its home cage.

The results for latency to platform in the Morris Water Maze test are presented graphically in FIG. 6 (Morris Water Maze Mean Time to Platform) in which error bars denote SEM. Block 1=B1, Block 2=B2, Normal Control (NC) n=11, Disease Control (DC) n=11, Acute pirfenidone (AC PFD) n=11, Chronic pirfenidone (CD PFD) n=12.

During water maze testing, an evaluation of initial learning processes showed a main effect for treatment overall (p=0.016, repeated measures ANOVA [block×treatment]), with post-hoc analysis revealing a statistically significant decrease in latency to platform for both acute and chronic pirfenidone treatment groups compared to Disease Controls (p=0.028, Disease vs Acute; p=0.052, Disease vs Chronic).

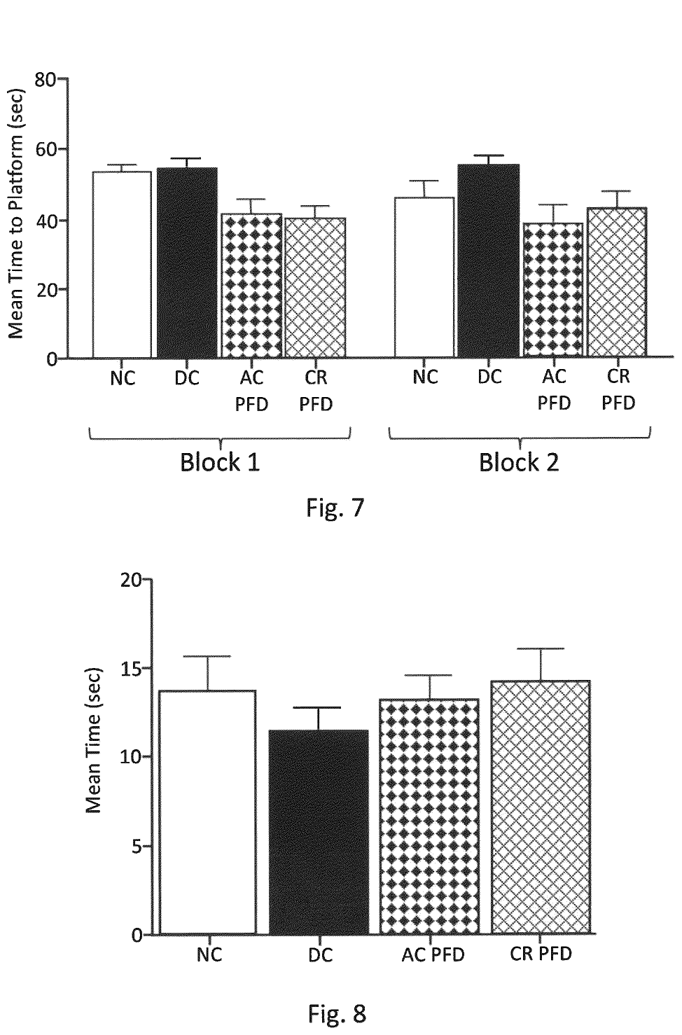

To provide a better understanding of the increase in initial learning noted in pirfenidone treated mice in this task, an evaluation of latency for each individual Block was performed (see FIG. 7) and showed a main effect during Block 1 testing (p=0.004, one-way ANOVA). Post-hoc analysis indicated a statistically significant decrease in latency to platform for mice that received Chronic pirfenidone treated mice compared to both Disease and Normal Controls (p=0.02 and p=0.03, respectively; Tukey's HSD post hoc), while mice in the Acute pirfenidone treatment group found the platform faster than Disease Controls only (p=0.04; Tukey's HSD post hoc). In Block 2, no between groups main effect was noted (p=0.117, one-way ANOVA; see FIG. 7). No difference between the pirfenidone treatment groups was noted, nor was there a difference between Normal and Disease Controls within Block (p>0.05).

To evaluate memory function in this test, paired t-tests were used. A decrease in latency between blocks provides an indication of spatial memory for platform location. Here, while statistical significance was not achieved, Normal Controls did exhibit a decrease in time to platform between blocks (p=0.12, paired t-test; Block 1 52.3 ±2.1 SEM, Block 2 45.5 ±5.1 SEM), a difference that was not seen in other groups (p=0.81, Disease Control; p=0.61, Acute pirfenidone; p=0.54, Chronic pirfenidone; paired t-tests).

Evaluation of swim speed showed no between group difference was present (p=0.273, one-way ANOVA).

Between groups differences did not achieve statistical significance during probe trial testing in the water maze task for time spent in the target quadrant or for swim speed (p>0.05, one-way ANOVA). However, it should be noted that, again, Disease Control animals exhibited decreased mean performance while treated mice behaved similarly to Normal Controls. Time in quadrant probe trial data is depicted graphically in FIG. 8.

### Evaluation of Hippocampal Microglial Cell Morphology

### Immunohistochemistry

A subset of mice was evaluated to compare acute and chronic treatment of pirfenidone paradigms against Normal Control and Disease Control tissues (n=8/treatment group). A neuroinflammation marker, ionizing calcium binding adaptor molecule 1 (Iba1), was investigated in the hippocampus as an indication of the level of inflammatory response. The hippocampus is particularly vulnerable to both acute and chronic stressors, including those triggered by physical trauma.

### Termination

Immediately following the final behavioral test, mice were deeply anaesthetized with isoflurane, cardiac blood was collected, mice were exsanguinated by transcardiac perfusion with normal PBS, and brain tissue was harvested.

### Brain Tissue Collection

Following saline perfusion, the brain was collected and divided into hemispheres. The right hemisphere was prepared for immunohistochemical evaluation.

### Protocol: Purpose: Materials:

To provide tissue for subsequent analysis
Dissection kit, cryovials/tubes
Saline
Liquid nitrogen
10% Neutral buffered formalin with 30% sucrose Optimal cutting temperature (OCT) media

### Cryopreservation of Right Hemisphere

1. Following dissection, cut the brain into two hemispheres with a razor blade.
2. Place the right hemisphere into 10% neutral buffered formalin with 30% sucrose.
3. Store at room temperature for 2-3 days to allow for complete fixation.
4. Orient brain with the cerebellum down, and freeze in OCT.
5. Store at −80° C.

### Histology

The right hemispheres of a subsection of study mice were evaluated via fluorescent IHC analysis to investigate the impact of laparotomy and drug intervention on hippocampal inflammatory mechanisms. Iba1 was assessed as a marker of neuroinflammation because it is indicative of inflammatory response in the central nervous system.

### Tissue Preparation

Cryoprotected hemispheres were blocked in OCT compound (Thermofisher) and stored at −80° C. until sectioning. Three coronal sections of 30 μm thickness were sectioned from the hippocampus from rostral, mid, and caudal regions using a Leica cryostat (chamber temperature-21° C.) into well plates containing Millonig's buffer and were stored at +4° C. until utilized for IHC analysis.

### Immunohistochemistry

### Heat-Induced Epitope Retrieval (HIER)

Scintillation vials containing either sodium citrate buffer for (10 mM tri-sodium citrate dihydrate, 0.05% Tween 20, pH adjusted to 6.0) were heated to 80° C. in a water bath. All tissues were incubated individually for 10 minutes, then allowed to cool for 10 minutes within the buffer before proceeding to the remainder of the immunohistochemical protocol.

### Immunohistochemistry: Two Day Protocol

Following HIER, tissues were permeabilized and blocked for two hours at room temperature, followed by overnight incubation at 4° C. in the appropriate primary antibody cocktail. The following day, sections were washed and incubated for 2 hours at room temperature in the appropriate secondary antibody cocktail, re-washed, mounted, coverslipped, and sealed.

### Procedure: Purpose: Materials:

### Protocol: Day 1:

Immunohistochemistry
To allow evaluation of Iba1 in brain tissue
Iba1 (1/2000 Abcam ab178846)
Donkey anti-rabbit 594 (1/2000 Invitrogen A21207)
0.3% Triton X-100 in PBS (PBS-T)
2% donkey serum+0.5% Triton X-100+0.1% sodium azide in PBS (blocking buffer) 1× PBS, Fluoromount G, coverslips, sealant
   1. Wash tissue three times in 1×PBS for 10 minutes each wash.
   2. Place tissue in 100° C. sodium citrate buffer for 10 minutes and allow to cool for an additional 10 minutes.
   3. Incubate in blocking buffer for 2 hours at room temperature.
   4. Incubate in primary antibody solution/blocking buffer overnight at 4° C. Protect from light.

### Day 2:

1. Wash sections three times in 1×PBS for 10 minutes each wash.
2. Incubate in secondary antibody solution/blocking buffer for 4 hours at room temperature. Protect from light.
3. Remove secondary by washing three times in 1×PBS for 5 minutes each wash.
4. Mount tissue onto slides, apply Fluoromount G, then coverslip and seal slides

### Imaging

### Fluorescent Microscopy

### Purpose: Materials:

### Procedure:

To obtain micrographs for quantification
Fluorescent microscope (Zeiss Axio Observer Z1) Apotome.2 imaging system Zeiss imaging software (Zen 2 Blue edition)
Settings were manually selected to allow visualization of signal on all sections (signal must be visible on low intensity sections but must not be overexposed on high intensity sections). Images were taken using a 20× objective, with tissue positioned to allow full visibility of the CA3/outer hippocampal region.

### Iba1 Quantification

Ionized calcium-binding adapter molecule 1 (Iba1) is a microglia-specific calcium-binding protein that participates in phagocytosis in activated microglia. It is specifically expressed in macrophages/microglia and is upregulated during the activation of these cells. Iba1 is thus a sensitive marker of inflammation and is quantified by measuring the intensity of the Iba1 positive signal.

Morphology of Iba1-stained microglia can provide a sensitive indication of cellular response to disturbances and thus function. Ramified microglia exhibit long, thin, branching projections with small cell bodies and are associated with homeostasis, while activated microglia have shorter and often thicker ramifications and a larger soma. Ameboid Iba1-stained microglia have cell bodies that are large and rounded, with no ramifications. Both activated and ameboid microglia are associated with phagocytotic processes and immune response.

Hippocampal tissue from the right hemispheres was evaluated for functional cell morphology using non-parametric rank ordering of tissue as follows:
0=normal morphology (ramified, mostly ramified)
1=moderate morphology (ramified-activated)
2=activated morphology (activated, activated ameboid, ameboid)

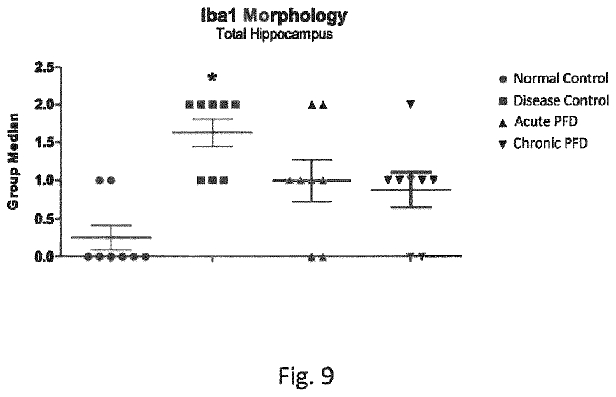

Morphological results from investigation of lba1-stained tissue at 8 days post-surgery are shown in FIG. 9. A non-parametric evaluation of Iba1-positive cell morphology revealed a statistically significant increase in Disease Control mice compared to Normal Controls for the expression of microglial cells exhibiting activated characteristics (p<0.05, Dunn's Multiple Comparison Test post-hoc). Both Acute and Chronic pirfenidone treatment reduced this microglial activation. The asterisk indicates a statistically significant difference from Normal Controls, while error bars denote SEM. All groups, n=8.

### Summary of Experimental Results

Overall, the results from the experiments described herein indicate that pirfenidone treatment exerts a protective effect for many of the behavioral changes associated with memory in postoperative cognitive dysfunction. During behavioral testing, Acute pirfenidone treated animals consistently exhibited responses equivalent to or better than those seen in Normal Controls, while Chronic pirfenidone administration also appeared to produce beneficial effects, particularly for recognition memory. At the cellular level, pirfenidone reduced microglial activation in the hippocampus. Pirfenidone is a promising solution to the problem of post-operative cognitive dysfunction, a long-recognized disorder for which adequate treatment is as yet unavailable and a long, unmet need.

### Pharmaceutical Compositions, Administration, and Dosages

It is contemplated that the pharmaceutical agents of the present invention can include pirfenidone alone or one or more (i.e., a combination) of pirfenidone, its analogs, and/or its derivatives. The analogs and derivatives of pirfenidone used in the present invention may be any of those disclosed in U.S. Pat. Nos. 6,090,822; 6,300,349; 6,956,044, for example. Each and all of these patents are incorporated by reference herein in the entirety.

In addition to pirfenidone (5-methyl-1-phenyl-2-1H-pyridine-2-one), it is contemplated that related molecules such as fluorofenidone (1-(3-fluorophenyl)-5-methylpyridin-2-one), Mefunidone [1-(4-((3-(4-methylpiperazin-1-yl) propyl)amino)benzyl)-5-(trifluoromethyl) pyridin-2(1H)-one], and other pirfenidone-related analogs and derivatives are of use in the present invention. These include N-substituted 2(1H) pyridones and N-substituted 3(1H) pyridines, which have been found useful in the treatment of disorders caused by excessive production of inflammatory cytokines.

The following are general structural formulas of N-substituted 2(1H) pyridones and N-substituted 3(1H) pyridones, which have the following structures:

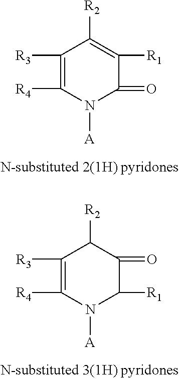

For the 2(1H) pyridones, R2 is an alkyl group and R3 is a hydrogen. Alternatively, R3 can be the alkyl group with R2 being a hydrogen. R1 and R4 are hydrogens.

For the 3(1H) pyridines, R2 is an alkyl group and R3 is a hydrogen. Alternatively, R3 can be the alkyl group with R1 being a hydrogen.

For both structures, A is typically an aryl group such as a phenyl, thienyl, etc.

It is contemplated that the pharmaceutical agents of the present invention may have ionizable groups such as, for example, —COOH and/or —CONH2, each of which could be attached, for example, to one or more of the R groups of the two N-substituted pyridones. These two ionizable groups are presented by way of illustration and are not meant in any way to be limiting. Molecules that have ionizable groups would also likely include the presence of a counter ion thus rendering the molecule a pharmaceutically acceptable salt. Common counter ions can include, but are not limited to, hydchloride, Sodium, sulphate, acetate, phosphate or diphosphate, chloride, potassium, maleate, calcium, citrate, mesylate, nitrate, tartrate, Aluminum, gluconate, etc. There can be certain benefits afforded by pharmaceutical salt formulations such as better solubility and/or improved gastrointestinal absorption, a change in drug half-life and metabolism, etc.

The following is an exemplary, but not necessarily limited, list of 2 and 3 pyridones:
5-Methyl-1-(3-nitrophenyl-2)-(1H) pyridone
5-Methyl-1-(4′-methoxyphenyl)-2-(1H) pyridone
5-Methyl-1-p-tolyl-2-(1H) pyridone
5-Methyl-1-(3′-trifluoromethylphenyl)-2-(1H) pyridone
1-(4′Chlorophenyl)-5-Methyl-2)-(1H) pyridone
5-Methyl-1-(2′-naphthyl)-2-(1H) pyridone
5-Methyl-1-(1′naphthyl)-2-(1H) pyridone
3-Methyl-1-phenyl-2-(1H) pyridone
3-Ethyl-1-phenyl-2-(1H) pyridone
6-Methyl-1-phenyl-2-(1H) pyridone
3,6-Dimethyl-1-phenyl-2-(1H) pyridone
5-Methyl-1-(2′-Thienyl)-2-(1H) pyridone
1-(2′-Furyl)-5-Methyl-2-(1H) pyridone
5-Methyl-1-(5′-quinolyl)-2-(1H) pyridone
5-Methyl-1-(4′-pyridyl)-2-(1H) pyridone
5-Methyl-1-(3′-pyridyl)-2-(1H) pyridone
5-Methyl-1-(2′-pyridyl)-2-(1H) pyridone
5-Methyl-1-(2′-quinolyl)-2-(1H) pyridone
5-Methyl-1-(4′-quinolyl)-2-(1H) pyridone
5-Methyl-1-(2′-thiazolyl)-2-(1H) pyridone
1-(2-Imidozolyl)-5-Methyl-2-(1H) pyridone
5-Ethyl-1-phenyl-2-(1H) pyridone
1-Phenyl-2-(1H) pyridone
1-(4′-Nitrophenyl)-2-(1H) pyridone
1,3-Diphenyl-2-(1H) pyridone
1-Phenyl-3-(4′-chlorophenyl)-2-(1H) pyridone
1,3-Diphenyl-5-methyl-2-(1H) pyridone
3-(4′-Chlorophenyl)-5-Methyl-1-phenyl-2-(1H) pyridone
5-Methyl-3-phenyl-1-(2′-thienyl)-2-(1H) pyridone
5-Methyl-1-phenyl-3-(1H) pyridone
5-Methyl-1-(4′-methoxyphenyl)-3-(1H) pyridone
5-Methyl-1-p-tolyl-3-(1H) pyridone
1-(4′-Chlorophenyl)-5-methyl-3-(1H) pyridone
5-Methyl-1-(2′-naphthyl)-2-(1H) pyridone
4-Methyl-1-phenyl-3-(1H) pyridone
6-Methyl-1-phenyl-3-(1H) pyridone
5-Methyl-1(2*-Thienyl)-3-(1H) pyridone
1-(2′-Furyl)-5-methyl-3-(1H) pyridone
5-Methyl-1-(5′-quinolyl)-3-(1H) pyridone
5-Methyl-1-(3′-pyridyl)-3-(1H) pyridone
5-Methyl-1-(2′-pyridyl)-3-(1H) pyridone
5-Methyl-1-(2′-quinolyl)-3-(1H) pyridone
5-Ethyl-1-phenyl-3-(1H) pyridone
1-Phenyl-3-(1H) pyridone

The pharmaceutical agents of the present invention can be administered before, during, and/or soon after surgery. They can be administered parenterally, intravenously, intra-arterially, intraperitoneally, intrathecally, intraventricularly, intrasternally, intracranially, intra-muscularly, transdermally, topically, subcutaneously, or by infusion. Preparing the agent for the above means of administration list is something that is well known to someone with ordinary skill in the art of sterile pharmaceutical compounding and administration formulations. For example, such a person would know which excipients, vehicles, buffers, salts, and/or other additional substances could be used to compound the pharmaceutical agents of the present invention and facilitate their administration to a patient.

The pharmaceutical agents of the present invention most typically may be administered by a surgeon or anesthesiologist to a patient in an appropriate formulation and at a time and dose that is consistent with the pharmacokinetics of the drug. The mean terminal half-life of pirfenidone is approximately 3 hours in healthy subjects. (https://www.accessdata.fda.gov/drugsatfda_docs/label/2017/208780s000lbl.pdf) Thus, for long operations that last several hours, it may be necessary to administer more than one dose of the drug or provide the drug in an extended-release formulation or continuous infusion.

For a mammal, it is preferred to use dosages of from about 10 to about 500 mg/kg body weight per day. For human patients, it is most preferred to use dosages of about 20 mg/kg to about 150 mg/kg per day. However, for human patients, dosage formulations can typically range from about 200 mg to 3,000 mg per dose.

Counter anions, such as potassium, sodium, calcium or zinc, for example may be used with the —COOH group, and hydrochloride may be used with the —CONH2 group.

The beneficial results demonstrated in this application are considered to be evidential of similar activity for all of the disclosed compounds and their pharmaceutically acceptable salts disclosed in this specification.

What is claimed is:

1. A method for the prevention and/or treatment of post-operative cognitive dysfunction, which comprises administering to a human or other mammal an effective amount of one or more compounds selected from the group consisting of N-substituted 2(1H) pyridones, N-substituted 3(1H) pyridones and pharmaceutically-acceptable salts of any one or more of the above, which are optionally further substituted at various available ring positions.

2. The method of claim 1, wherein for the N-substituted 2(1H) pyridones R1 and R4 are hydrogen, R2 is a C1-C10 alkyl group which is optionally substituted, and R3 is hydrogen; or R2 is hydrogen and R3 is a C1-C10 alkyl group which is optionally substituted.

3. The method of claim 1, wherein for the N-substituted 3(1H) pyridones R2 is a C1-C10 alkyl group which is optionally substituted, and R3 is hydrogen; or
R3 is a C1-C10 alkyl group which is optionally substituted and R1 is hydrogen.

4. The method of claim 2, wherein the C1-C10 groups of R2 and/or R3 are substituted with fluoro groups.

5. The method of claim 3, wherein the C1-C10 groups of R2 and/or R3 are substituted with fluoro groups.

6. The method of claim 1, wherein a dosage from about 10 mg to about 500 mg/kg of body weight per day of one or more of said compounds in total.

7. The method of claim 6, wherein a dosage of from about 20 mg to about 150 mg/kg of body weight per day of one or more of said compounds in total.

8. The method of claim 1, wherein administration is about 200 mg to 3,000 mg per dose to humans.

9. The method of claim 1, wherein said one or more compounds and/or salts thereof are administered before, during or after surgery or any combination thereof.

10. The method of claim 9, wherein said one or more compounds of salts thereof are administered as an extended-release formulation before surgery or by continuous infusion during surgery

10. The method of claim 1, wherein said one or more compounds administered are pirfenidone or a pharmaceutically-acceptable salt thereof.

11. The method of claim 10, wherein said pharmaceutical salt of said one or more compounds comprise an inorganic salt of a —COOH group or a —CONH2 group.

12. The method of claim 11, wherein said salt of said-COOH group is with a pharmaceutically-acceptable counter ion, comprising potassium, sodium, calcium or zinc cations.

13. The method of claim 11, wherein said salt of said —CONH2 group, protonated, is with a pharmaceutically acceptable counter ion comprising hydrochloride.

14. The method of claim 1, wherein said one or more compounds and/or pharmaceutically acceptable salts thereof is administered in a form selected from the group consisting of capsules, tablets, powders, granules, syrups, aerosols, injectable fluids, intravenous fluids, pills, creams, ointments, inhalable fluids, eye drops and suppositories.

15. The method of claim 1, wherein said one or more compounds or pharmaceutically-acceptable salts thereof reduce microglial activation in the hippocampus.

16. The method of claim 1, wherein the N-substituted 2(1H) and 3(1H)-pyridones comprise:
5-methyl-1-(3-nitrophenyl-2)-(1H) pyridine
5-methyl-1-(4′-methoxyphenyl)-2-(1H) pyridine
5-methyl-1-p-tolyl-2-(1H) pyridine
5-methyl-1-(3′-trifluoromethylphenyl)-2-(1H) pyridone
1-(4′-chlorophenyl)-5-methyl-2)-(1H) pyridone
5-methyl-1-(2′-naphthyl)-2-(1H) pyridone
5-methyl-1-(1′-naphthyl)-2-(1H) pyridone
3-methyl-1-phenyl-2-(1H) pyridone
3-ethyl-1-phenyl-2-(1H) pyridone
6-methyl-1-phenyl-2-(1H) pyridone
3,6-dimethyl-1-phenyl-2-(1H) pyridone
5-methyl-1-(2′-thienyl)-2-(1H) pyridone
1-(2′-furyl)-5-methyl-2-(1H) pyridone
5-methyl-1-(5′-quinolyl)-2-(1H) pyridone
5-methyl-1-(4′-pyridyl)-2-(1H) pyridone
5-methyl-1-(3′-pyridyl)-2-(1H) pyridone
5-methyl-1-(2′-pyridyl)-2-(1H) pyridone
5-methyl-1-(2′-quinolyl)-2-(1H) pyridone
5-methyl-1-(4′-quinolyl)-2-(1H) pyridone
5-methyl-1-(2′-thiazolyl)-2-(1H) pyridone
1-(2-imidazolyl)-5-methyl-2-(1H) pyridone
5-ethyl-1-phenyl-2-(1H) pyridone
1-phenyl-2-(1H) pyridone
1-(4′-nitrophenyl)-2-(1H) pyridone
1,3-diphenyl-2-(1H) pyridone
1-phenyl-3-(4′-chlorophenyl-2-(1H) pyridone
1,3-diphenyl-5-methyl-2-(1H) pyridone
3-(4′-chlorophenyl-5-methyl-1-phenyl-2-(1H) pyridone
5-methyl-3-phenyl-1-(2-thienyl)-2-(1h) pyridone
5-methyl-1-phenyl-3-(1H) pyridone
5-methyl-1-(4′-methoxyphenyl-3-(1H) pyridone
5-methyl-1-p-tolyl-3-(1H) pyridone
1-(4′-chlorophenyl)-5-methyl-3-(1H) pyridone
5-methyl-1-(2′-napthyl)-2-(1H) pyridone
4-methyl-1-phenyl-3-(1H) pyridone
6-methyl-1-phenyl-3-(1H) pyridone
5-methyl-1-(2′-thienyl)-3-(1H) pyridone
1-(2′-furyl)-5-methyl-3-(1H) pyridone
5-methyl-1-(5′-quinolyl)-3-(1H) pyridone
5-methyl-1-(3′-pyridyl)-3-(1H) pyridone
5-methyl-1-(2′-pyridyl)-3-(1H) pyridone
5-methyl-1-(2′-quinolyl)-3-(1H) pyridone
5-ethyl-1-phenyl-3-(1H) pyridone, or
1-phenyl-3-(1H) pyridone, or any combination thereof.
